(19) United States
(12) Patent Application Publication
(10) Pub. No.: US 2026/0053652 A1
(43) Pub. Date: Feb. 26, 2026

(54) A METHOD AND SYSTEM FOR DESIGNING ORTHOSES

(71) Applicant: FOOT CARE LTD., Rishone leZion (IL)

(72) Inventors: Adi SAMSONOFF, Rishone leZion (IL); Philip WELLS, Staffordshire (GB)

(21) Appl. No.: 19/104,223

(22) PCT Filed: Aug. 16, 2023
(86) PCT No.: PCT/IL2023/050864

§ 371 Date: Feb. 16, 2025

(30) Foreign Application Priority Data
Aug. 16, 2022 (IL) 295690

(51) Int. Cl. A61F 5/01; A61B 5/107; G06T 17/00; G06T 19/20

(52) U.S. Cl. CPC A61F 5/0111; A61B 5/1074; G06T 17/00; G06T 19/20

(57) ABSTRACT

A method and computer program product, the method comprising: obtaining a 3D computerized foot model of a foot of a patient; displaying the 3D computerized foot model over a display device; receiving from a user locations of a first set of model points on the 3D computerized foot model, each model point of the first set associated with predetermined anatomical features; automatically adapting a base shell 3D model upon the first set of model points to obtain an adapted shell model; automatically calculating an additional set of model points based on the first set of model points, the first set and the additional set of model points representing a shape of a foot arch of the patient; and automatically enhancing the adapted shell model upon the first set of model points and the additional set of model me points to obtain an orthosis deign for the foot.

### TECHNICAL FIELD

The present disclosure relates to orthoses in general, and to a method and apparatus for designing and generating personally-adapted orthoses, in particular.

### BACKGROUND

An orthosis is an externally applied device, usually used to modify the structural and functional characteristics of the neuromuscular and skeletal system. An orthosis may be used to support, control, guide, limit and/or immobilize an extremity, joint or body segment for a particular reason. An orthosis may assist in the movement of a particular body part, for example by restricting the movement in a given direction, changing the weight distribution affected by stepping, reducing weight bearing forces for a particular purpose, aiding in rehabilitation from fractures after the removal of a cast, or otherwise correcting the shape and/or function of the body, to provide easier movement capability or reduce pain.

A lower-limb orthosis is an external device applied to a lower-body segment to improve function, by controlling motion, providing support through stabilizing gait, reducing pain through transferring load to another area, correcting flexible deformities, or preventing progression of fixed deformities. Lower-limb orthoses include various types of ankle-foot orthoses, and foot orthoses.

Foot orthoses are devices inserted into patients' shoes to provide support for the foot by redistributing ground reaction forces acting on the foot joints while standing, walking or running. Orthoses can have significant impact on foot, knee, hip, and spine deformities, and are aimed at aiding in a wide range of biomechanical deformities and a variety of soft tissue conditions, such as but not limited to painful high-arched or low-arched feet, and may be effective for people with rheumatoid arthritis, plantar fasciitis, hallux valgus (“bunions”) or other problems.

Some orthoses are pre-molded (also referred to as pre-fabricated), while others are custom made, traditionally according to a cast or impression of the foot. If the orthosis fits the patient's foot well, it can have a highly positive impact, while a poorly fitted orthosis can worsen the patient's situation, cause pain and walking difficulties, or the like. Thus, it is important that the orthosis is well fitted to the patient's foot.

Traditionally, orthoses are generated by experts adapting ready shells in accordance with a three dimensional cast or impression of the foot, using their knowledge and experience. However, very often an orthosis need to be corrected either due to initial mismatch and discomfort of the patient, or to changes caused by repeated usage.

### BRIEF SUMMARY

One exemplary embodiment of the disclosed subject matter is a computer-implemented method for generating an orthosis shell design, the method comprising: obtaining a three dimensional (3D) computerized foot model of a foot of a patient; displaying the 3D computerized foot model over a display device; receiving from a user locations of a first set of model points on the 3D computerized foot model, each model point of the first set of model points associated with predetermined anatomical features; automatically adapting a base shell 3D model upon the first set of model points to obtain an adapted shell model; automatically calculating an additional set of model points based on the first set of model points, the first set of model points and the additional set of model points representing a shape of a foot arch of the patient; and automatically enhancing the adapted shell model upon the first set of model points and the additional set of model points to obtain an orthosis deign for the foot. The method can further comprise: upon receiving from the user an indication of a segment across the foot model, displaying a first cross section of the adapted shell model as enhanced and a second cross section of the 3D computerized foot model, for which the projections over a plane of the display device aligns with the segment. The method of claim 1, further comprising: receiving from the user an updated location for a point from the first set of model points or from the additional set of model points; and updating the adapted shell model in accordance with the updated location. The method can further comprise: receiving from the user an updated value for a pre-defined parameter for controlling a shape of the base shell 3D model or the adapted shell model; and updating the adapted shell model in accordance with the updated value. Within the method, the pre-defined parameter is at least one item selected from the group consisting of: scale, heel width, forefoot width, medial trim line, lateral trim line, raising of heel cup, and thickness. Within the method, the first set of model points optionally includes at least one point selected from the group consisting of: a first metatarsal point (point a), a fifth metatarsal point (point b) and a heel center (point c). Within the method, an x coordinate and a y coordinate of the additional set of model points are optionally determined based on x and y coordinates of point a, point b and point c, and wherein a height (z coordinate) is determined by ray casting to indicate a point on the 3D computerized foot model. Within the method, calculating the additional set of model points optionally includes resizing and aligning the base shell 3D model and the additional set of model points optionally describes an arch of the foot. Within the method, the additional set of model points optionally includes 5 points, being point 0, point 1, point 2, point 3 and point 4. Within the method, the additional set of model points optionally includes at least one point selected from the group consisting of: a midpoint between point a and a point having an x coordinate of point a and a y coordinate of point c (point 4); a midpoint between point a and point 4 (point 2); a center point between point b and point 4 (point 3); a point having a y coordinate of point 4 and an x coordinate of point c (point 0); and a midpoint between point 0 and point 4 (point 1). Within the method, enhancing the adapted shell model optionally comprises: using point c as a reference point, determining resizing and aligning parameters for the base shell 3D model to comply with the 3D computerized foot model based on the locations of point a and point b relative to the 1^{st} and 5^{th} metatarsal points on the base shell 3D model, and adapting the base shell 3D model; determining x and y coordinates for additional shell points on the base shell 3D model as adapted, using the resizing and aligning parameters as applied to the x and y coordinates of the additional set of model points; updating the base shell 3D model as adapted such that a z coordinate of each of the additional shell points corresponds to the z coordinate of a corresponding model point from the additional set of model points; and enhancing the base shell 3D model as updated in a fall off area around at least one of the additional set of model points. Within the method, the base shell 3D model is optionally in compliance with an orthosis model selected by the user. The method can further comprise generating an orthosis based on the orthosis design. Within the method, the 3D computerized foot model is optionally generated by scanning a foot or a foot model or a foot impression. Within the method, the base shell 3D model is optionally selected from a shell library comprising basic shell designs, wherein the base shell 3D model is a data structure enabling said adapting and said enhancing. The method can further comprise storing the first set of model points, the additional set of model points and the adapted shell 3D model; and retrieving the first set of model points, the additional set of model points or the adapted shell 3D model for performing a future task associated with the patient. The method can further comprise: obtaining a second 3D computerized foot model of a second foot of the patient; displaying the second 3D computerized foot model over a display device; receiving from a user second locations of the first set of model points on the second 3D computerized foot model; comparing the locations and the second locations while taking into account their symmetry to obtain a correspondence degree; subject to the correspondence degree being acceptable, creating a second orthosis deign for the second foot, the second orthosis deign being a symmetrical reflection of the orthosis design. The method can further comprise: subject to the correspondence degree being unacceptable, generating the second orthosis design, and upon receiving from the user an indication of a segment across the foot model, displaying a first cross section of the orthosis design and a second cross section of the second orthosis, for which the projections over a plane of the display device aligns with the segment.

Another exemplary embodiment of the disclosed subject matter is a computerized apparatus having a processor, the processor being adapted to perform the steps of: obtaining a three dimensional (3D) computerized foot model of a foot of a patient; displaying the 3D computerized foot model over a display device; receiving from a user locations of a first set of model points on the 3D computerized foot model, each model point of the first set of model points associated with predetermined anatomical features; automatically adapting a base shell 3D model upon the first set of model points to obtain an adapted shell model; automatically calculating an additional set of model points based on the first set of model points, the first set of model points and the additional set of model points representing a shape of a foot arch of the patient; and automatically enhancing the adapted shell model upon the first set of model points and the additional set of model points to obtain an orthosis deign for the foot.

Yet another exemplary embodiment of the disclosed subject matter is a computer program product comprising a computer readable storage medium retaining program instructions, which program instructions when read by a processor, cause the processor to perform a method comprising: obtaining a three dimensional (3D) computerized foot model of a foot of a patient; displaying the 3D computerized foot model over a display device; receiving from a user locations of a first set of model points on the 3D computerized foot model, each model point of the first set of model points associated with predetermined anatomical features; automatically adapting a base shell 3D model upon the first set of model points to obtain an adapted shell model; automatically calculating an additional set of model points based on the first set of model points, the first set of model points and the additional set of model points representing a shape of a foot arch of the patient; and automatically enhancing the adapted shell model upon the first set of model points and the additional set of model points to obtain an orthosis deign for the foot.

### THE BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1 shows a flowchart of steps in a method for designing and manufacturing an orthosis model for a patient, in accordance with some exemplary embodiments of the disclosed subject matter;

FIG. 2 shows a flowchart of steps in a method for designing a shell model based on a first set of points provided by the user, in accordance with some exemplary embodiments of the disclosed subject matter;

FIG. 3A shows a foot model as displayed over a display device, in accordance with some exemplary embodiments of the disclosed subject matter;

FIG. 3B shows the foot model of FIG. 3A, with two marked user-provided points, in accordance with some exemplary embodiments of the disclosed subject matter;

FIG. 3C shows the foot model of FIG. 3A as rotated with three marked user-provided points, and the additional set of points, in accordance with some exemplary embodiments of the disclosed subject matter;

FIG. 4A and FIG. 4B show two additional views of the foot model with three marked user-provided points and the additional set of points, in accordance with some exemplary embodiments of the disclosed subject matter;

FIG. 5A shows a top view of the foot model and the shell model, and a line across the models representing a cross section, in accordance with some exemplary embodiments of the disclosed subject matter;

FIG. 5B shows cross sections of the foot model and the shell model corresponding to the line of FIG. 5A, in accordance with some exemplary embodiments of the disclosed subject matter;

FIGS. 6A and 6B show different views of the foot model and the shell model, in accordance with some exemplary embodiments of the disclosed subject matter;

FIG. 7A shows models of the right and left feet of a patient, in accordance with some exemplary embodiments of the disclosed subject matter;

FIG. 7B shows a combined model of the feet and the differences, in accordance with some exemplary embodiments of the disclosed subject matter; and

FIG. 8 shows a block diagram of an apparatus for designing personally fitted orthoses, in accordance with some exemplary embodiments of the disclosed subject matter.

### DETAILED DESCRIPTION

Orthotics, and in particular foot orthotics can significantly affect a patient's state, and specifically foot, knee, hip, and spine deformities, and can thus change for better or worse a patient's neuromuscular system while standing, walking, running or performing other activities.

In the description below, unless noted otherwise, the term patient refers to a person for whom orthoses are being designed and generated. The term user refers to a professional such as a podiatric, orthosis expert or another person involved in the process of designing and generating orthoses for patients.

One technical problem of the subject matter is the need to design and generate a personal orthosis for a patient, in accordance with the specific structure of the patient's foot and the intended use of the orthosis, such that the orthosis will provide support where needed, flexibility where needed, adapt the height at different areas of the foot, or the like.

However, currently available computerized systems cannot fully replace an experienced professional who has designed hundreds, thousands or more orthoses, and has learned from the experience gathered through interacting and receiving feedback from multiple patients. Neither can the computerized system utilize verbal input from a patient describing the patient's needs, pains, limitations, or the like. On the other hand, designing an orthosis by an expert is a labor and time consuming process. Although many parts of the process are repetitive and need to be done for almost all patients with little changes, they still need to be performed for each patient separately, and thus take a lot of time and significantly limit the capacity of the expert. Moreover, when generating a new orthosis for the same patient, the whole process needs to be repeated.

Moreover, even in sophisticated computerized systems, it is hard to introduce a local change to an orthosis being designed, while maintaining the smoothness of the orthosis and avoiding sharp protrusions or other discomforts.

Another technical problem of the subject matter relates to the practical process of providing an orthosis to a patient. It may be a significant burden for the patient, and in particular a patient living far away from a clinician, an old patient, or a patient with serious disabilities to arrive to a clinic, have their feet measured or imprinted, and then come back to pick the orthosis once ready.

One technical solution comprises an orthosis designing system, that receives a three dimensional (3D) computerized model of a patient's foot, and with just a few actions by an expert using a dedicated software, generates a personally-adapted orthosis, suitable for the patient.

Thus, a patient's foot may be scanned, for example with a camera, a video camera, a smartphone, or the like. Alternatively, an imprint box in which the patient has imprinted his foot may be scanned by a camera. The scan may include capturing two or more still images or a stream of video frames, and a model of the foot may be generated, as disclosed, using any 3D modeling technique currently known or that will become known in the future, The user may then inspect the model on a computerized platform, and may indicate a set of model points having anatomic significance. For example, the user may select the 1st metatarsal point, the fifth metatarsal point and the heel center.

The system may then automatically select a set of additional points on the model based on the first set of points, such that the set of additional points represent an anatomical feature, such as the foot arch.

A predetermined orthosis model, also referred to as base shell, may then be obtained according to the user's selections such as size, color, or the like. The term base shell refers to the default basic shape of a style of orthotics, such as a ¾ shell (a shell that supports only the back ¾ of the foot, and not the toes area), a full-length shell, or the like. The orthosis model, having indicated thereon points corresponding to the points selected by the user, may then be resized according to the distances between the points of the first set, such that its size is in accordance with the size of the foot model. Subsequently, points on the orthosis model corresponding in two dimensions to the additional set of points as determined by the system, may be located, and their third dimension may be adapted such that they represent points that are located on the foot model, and hence on the patient's foot. In addition to adapting the specific points in accordance with the additional set of points, the area surrounding each such point may also be adapted, thereby altering the base shell to obtain a smooth shell model adapted to the patient's foot.

The user may correct the location of one or more of the first set of points, or one or more of the additional points, and the model may be adapted accordingly, while maintaining its smoothness.

The user may also adapt other parameters of the shell, such as general scale, heel width, forefoot width, adjustments to the medial/lateral trim line, raising of the heel cup, thickness definition, or others. The user may also adapt the orthosis based on verbal or other input from the patient, the patient's physician or another expert. For example, some instructions may accompany the model, and be considered by the user.

In addition to presenting the foot model, the orthosis model, the first set of points as indicated by the user, and the additional points as calculated by the system, the system may also enable the user to mark any straight line over the displayed foot model. The system may then present cross sections of the foot model and of the orthosis along the straight line.

The foot model, the selected points and other changes introduced by the user may be stored, such that another orthosis may be generated for the patient with minimal or no changes.

The orthosis model may then be provided to a manufacturing facility for production. In some embodiments, the facility may be located close to the patient's location, to reduce shipping costs.

One technical effect of the disclosure relates to a hybrid system and method for designing an orthosis for a patient, wherein the design combines minimal but effective user input in the form of identifying key anatomical points, followed by using these points for automatic generation of a smooth orthosis model, suitable for the patient and especially adapted to the patient's foot arch. The design may then be further adapted at certain areas in accordance with the user's expertise or input from the patient.

Another technical effect of the disclosure relates to the option to start the process with a well-defined shell that fits the patient's size, enhancing the shell automatically based on the patient's anatomical features, and followed by introducing local but smooth enhancements to the design, while maintaining the general shape of the arch.

Yet another technical effect of the disclosure relates to the option to store a minimal amount of data which enables to reconstruct the orthosis model for introducing changes, generating another orthosis, or the like.

Yet another technical effect of the disclosure relates to minimizing physical object production, packaging and shipping. Thus, the foot model may be generated upon digital scan rather than a physical imprint which needs to be shipped to the expert. Additionally or alternatively, the final orthosis design which is in a digital form may be transmitted to a production facility close to the patient, and thus further reduce the shipping costs.

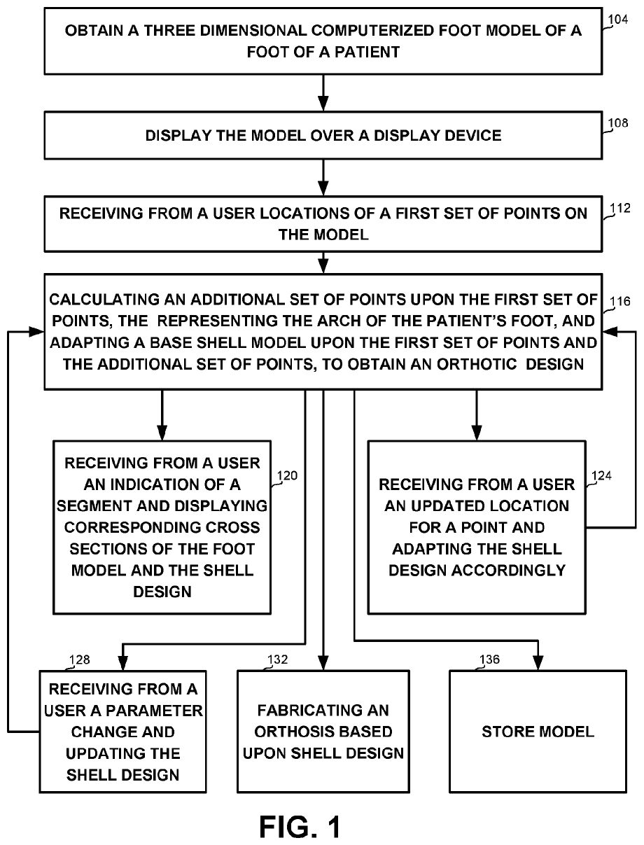

Referring now to FIG. 1, showing a flowchart of steps in a method for designing and manufacturing an orthosis model for a patient, in accordance with some embodiments of the disclosure.

On step 104, a three dimensional (3D) computerized model of the user's foot may be obtained. The model may be for example in STL, OBJ, STX format, or any other format suitable for describing 3D objects. The model may be generated from two or more images of the foot, from an imprint of the foot in a box filled with material adapted to capture and maintain the foot shape, from images of the imprint, or in any other manner, using for example any 3D scanning technique.

In addition to the model, detailed information may be received from the patient or another person such as the patient's caregiver, regarding relevant diagnosis, pains or other complaints, intended use for the requested orthosis, or the like.

The model may be provided to a user such as an orthotics expert, a podiatrist, or the like over a communication network, on a physical storage device, by e-mail, retrieved from a database, or the like.

On step 108, the model may be displayed to the user. The model may be displayed over a display device, such as a computer screen, a touch screen, or the like.

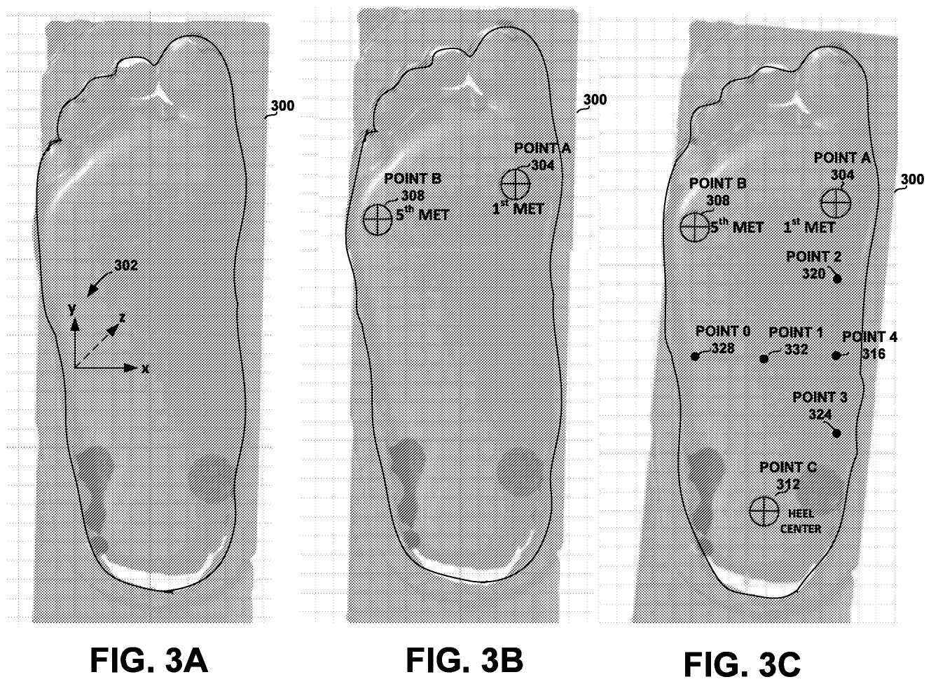

FIG. 3A shows foot model 300 as displayed within a user interface of a 3D editing software over a display device. The user may rotate the view in any direction to see other areas and other projections of the model, scale the view or manipulate it in any other manner.

On step 112, a first set comprising three or more model points may be received from the user, indicating predetermined anatomical locations, such that they are identifiable by an expert. For example, the points may include the 1st metatarsal point, the 5th metatarsal point, and the heel center. Although these specific points can be identified in a two dimensional projection of the foot model, the user may rotate the view to further examine and possibly change the locations of the marked points.

FIG. 3B shows view 300 with two marked user-provided points, being the 1st metatarsal point 304 (referred to as point a) and the 5th metatarsal point 308 (referred to as point b).

FIG. 3C further shows view 300 with point a 304, point b 308, and heel center 312 (referred to as point c).

It will be appreciated that the three points are merely an example, and any different set of points which can be identified from the foot model may be used.

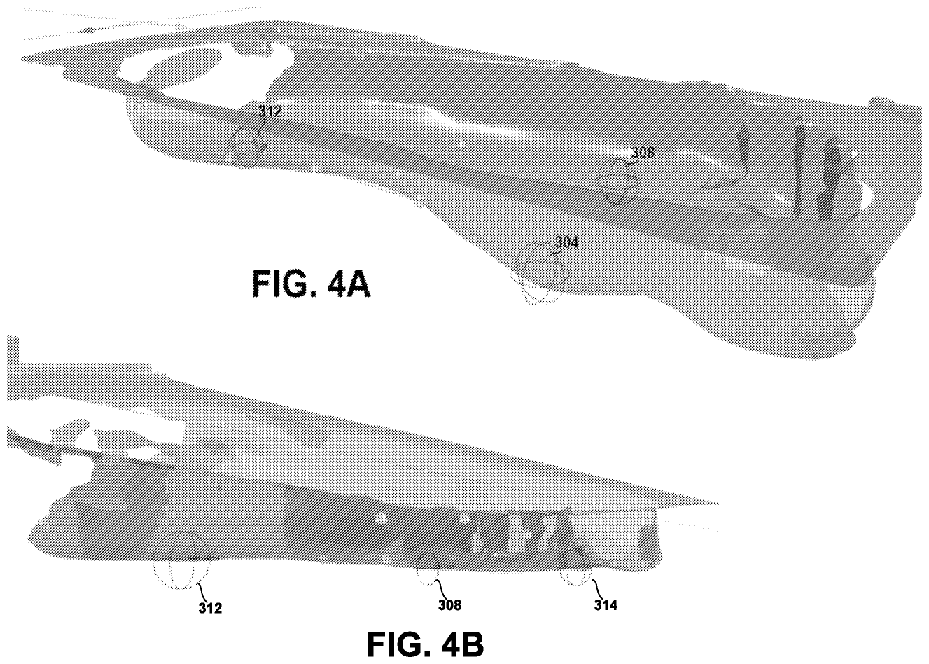

FIGS. 4A and 4B show different views of the 3D foot model with the three points marked thereon.

It will be appreciated that the user can mark the points in any order. Upon marking a point, the user may be asked to indicate which of the three points (1st metatarsal point, 5th metatarsal point and heel center) he is currently marking.

Once the user has marked the third point, step 116 may take place.

Step 116 may include automatically calculating an additional set of points based on the model points selected by the user, wherein the combination of the additional points has anatomical significance. For example, the set of additional points can approximate the shape of the user's foot arch, which is one of the most, if not the most important feature of an orthosis, and the one that varies most between patients. For example, the points can approximate how high or low, narrow or wide the foot arch is, where is its highest point, and additional parameters of the foot arch. Then, based on the model points selected by the user, and the additional set of points, a base shell 3D model may be automatically adapted to the user's foot shape.

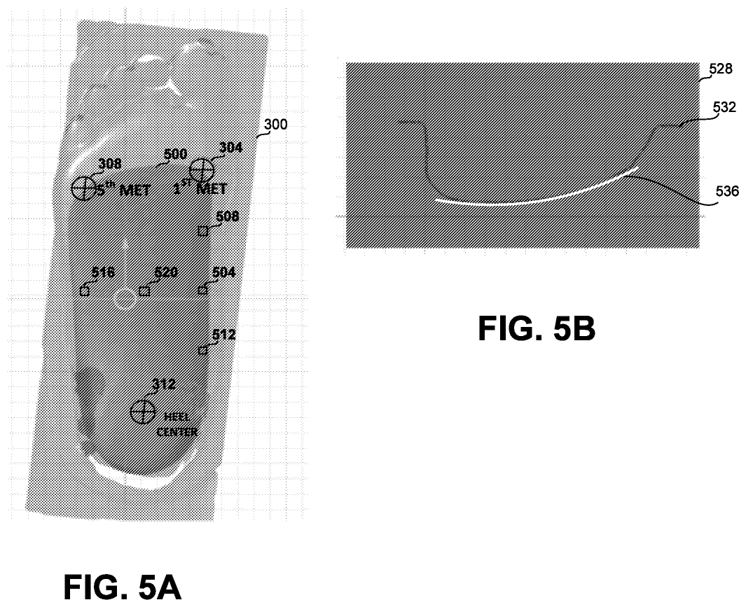

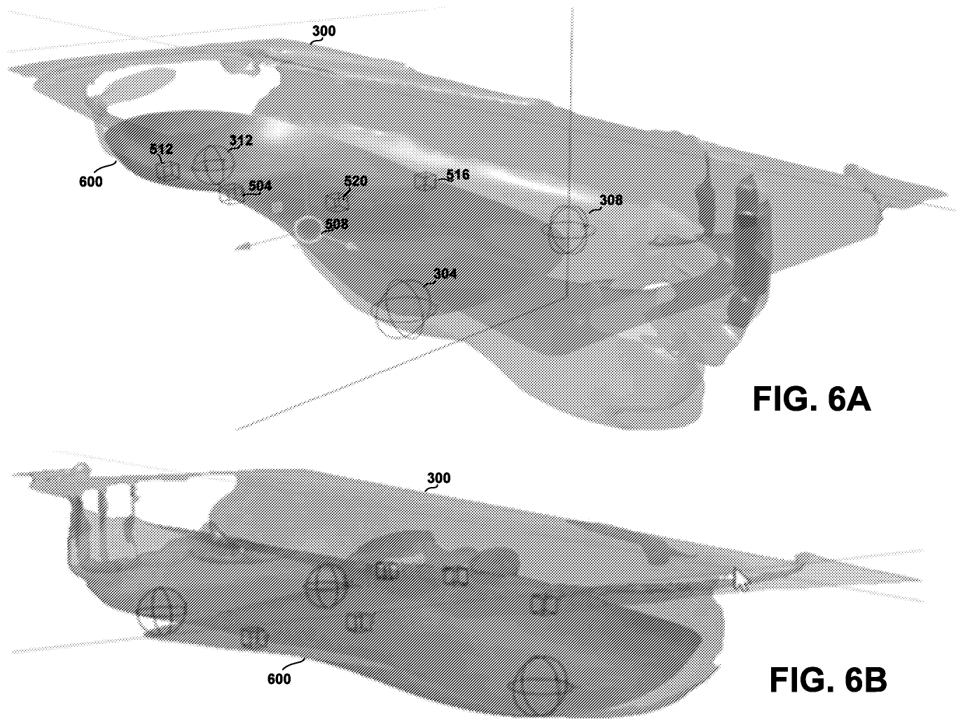

FIG. 5A and FIG. 6 show different views of the foot model, with the first set of points 304, 308 and 312, and the additional points 504, 508, 512, 516 and 520 marked thereon.

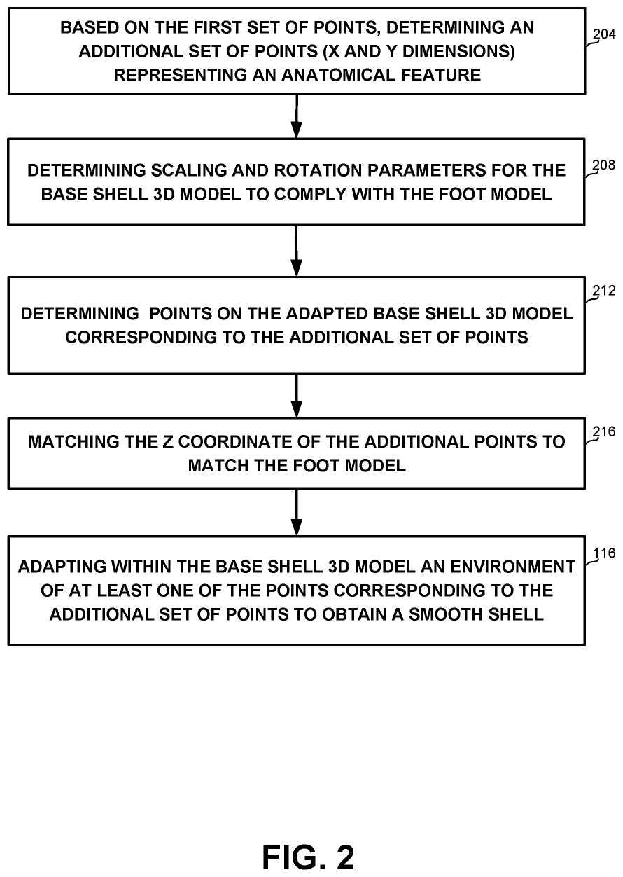

The determination of the additional points and adaptation of the base shell 3D model for obtaining a shell model is further detailed in association with FIGS. 2, 5A, 6A and 6B further detailed below.

Once completed, or at any earlier stage, the shell model may be stored. It may not be required to store the full shell model. Rather, storing only the selected base shell 3D model and its parameters, the first step of points and the amendments introduced by the user may be sufficient for resuming the process, designing another shell model for the same patient, amending the current shell model upon patient's feedback, or the like.

Once the base shell 3D model is available, it may be viewed, assessed, manipulated or used in a variety of ways.

In one example, on step 120, an indication of a line crossing the displayed foot model or shell model may be received from the user, and cross sections of the foot model and of the shell model as designed may be displayed. For example, line 524 of FIG. 5A marked over foot model 300 and shell model 500 being designed is marked by the user. In response, as shown in FIG. 5B graph 528 is displayed, showing cross section 532 of the foot model and cross section 536 of the designed shell, such that the user can view and adjust the shell model to fit the foot model and the needs. As the user moves line 524, cross sections 532 and 536 may change accordingly. The cross sections may be used as an assessment tool for the user, helping the user asses how well the orthosis fits the foot arch, and whether or not further shape manipulations are required.

In another example, on step 124 an updated location to any of the first set of points may be received from a user. The user may change the selection of the points within the foot model in the 3D space in a graphic manner, by updating their coordinates through typing text, or the like. The user may also be able to change the location of one or more of the additional points, such as point 4 or point 0. Any of these changes may cause a change in one or more of the other points in the additional set of points, and thus in the shape of the designed shell model and in particular in the area of the shell model corresponding to the user's foot arch. Once the points are changed, execution may return to step 116 for adapting the shell model accordingly. It is appreciated that execution may return to different stages of the shell model design as detailed in association with FIG. 2, depending on whether the user has changed one of the first set of points, or one of the additional set of points.

In some embodiments, if the user changes the location of one or more of the additional set of points, such change may cause an update of the respective point in of the shell model, and its surrounding area, to ensure the smoothness of the shell.

In yet another example, on step 128 a change to one or more parameters of the shell model may be received from the user. For example, the user may change the shell type, size, width, or other parameters of the shell. The user may also change the shape, radius or another parameter of the area around each point of the additional set of points that is affected by changing the point itself, referred to as the fall off area. Once a parameter is changed, execution may return to step 116 for adapting the shell design accordingly. It is appreciated that execution may return to different stages of the shell design as detailed in association with FIG. 2, depending on which parameter of which change has been introduced.

It is appreciated that when designing the shell model, the user may also consider data provided by the patient or a person on his behalf, the data provided with the patient's foot model, on a conversation between the user and the patient, or the like. Thus, the user may enhance the shell in accordance with the patient's specific complaints or requests, for improved fitting of the orthosis.

In yet another example, on step 132 the shell model as designed may be provided to a manufacturing facility for production, and the physical orthosis produced according to the shell design may be shipped to the patient. Fabrication may also be based on patient requests, related for example to the color or other parameters.

In yet another example, on step 136 the shell model as designed may be stored, optionally together with additional data, such as but not limited to data regarding the specific shell design. In some embodiments, minimal data is required to be stored, including the shell type, parameters, first set of points and the introduced changes.

The shell structure and calculations may be performed under any required computational approach, such as but not limited to a polygonal 3D approach, surface modeling approach, CAD, NURBS, or others.

Referring now to FIG. 2, showing a flowchart of steps in a method for generating a shell model based on the first set of points provided by the user, in accordance with some exemplary embodiments of the disclosure.

On step 204, an additional set of points may be determined, based upon the first set of points.

In the discussion below it is assumed that the y dimension of the foot model is along the patient's foot length heel-to-toe, its x dimension is along the foot width side-to-side, and the z dimension is the foot height (parallel to with the patient's head-to-foot axes). A projection, such as a top view projection of the foot model over the x-y plane may be initially presented to the user.

The foot model may then be rotated on the x-y plane using the heel center point 312 as an axis, such that the x coordinate of the heel center point 312 is the average of the x coordinates of the 1st metatarsal point 304 and the 5th metatarsal point 308, as shown in FIG. 3C.

Once the model is aligned as described above, the additional points may be selected as follows, and as shown on FIG. 3C:

A segment parallel to the y axis is indicated between the 1st metatarsal point 304, and a point having the same y coordinate as heel center point 312.

The middle point of this segment is referred to as point 4 (316).

The middle point between point 4 (316) and the 1st metatarsal point 304 is referred to as point 2 (320).

The middle point between point 4 (316) and the point having the same y coordinate as heel center point 312 and the same x coordinate as point 4 (316) is referred to as point 3 (324).

Point 0 (328) is indicated as a point having the same x coordinate as 5th metatarsal point 308 and the y coordinate of point 4 (316).

Point 1 (332) is indicated as a middle point between point 0 (328) and point 4 (316).

It will be appreciated that the points may also be selected without rotating the foot model, and the rotation is described and executed for making the computations easier and the explanation above clearer.

On step 208, for a given base shell, resizing and aligning parameters may be determined, for adjusting the base shell according to the first point. It will be appreciated that the base shell 3D model may be a default model type. Additionally or alternatively, the base shell 3D model type may be selected by the user from a variety of shells, such as a full length orthosis, a ¾ length orthosis, or the like. Each such shell may be associated with different default parameters.

For each shell type, the 1st and the 5th metatarsal points and the heel center are pre-indicated. By using one of the points, for example the heel center point, as a pivot and matching the other two points to the corresponding points on the foot model, the resizing and aligning transformations between the foot model and the base shell may be determined, thereby obtaining a shell model adapted in size to the patient's foot.

On step 212, points are determined on the shell model, which have x and y coordinates that correspond to the additional set of points of the foot model, as resized and aligned in accordance with the resizing and aligning parameters. Thus, these additional points keep the same proportions among them and with the first set of points in the x-y plane over the shell model.

On step 216, the z coordinate of each of the additional points is determined to match the z coordinate of the respective point on the foot model. The Z coordinate of the additional points may be calculated, for example, using ray casting, to position each of the additional points such that it marks a point on the foot model.

FIGS. 6A and 6B show different views of foot model 300 together with the shell model 600, the first set of points and the additional set of points.

On step 220, a surrounding area, referred to as “fall off” area around each of the additional set of points may be adapted to ensure that the resulting shell is smooth. Default values for the shape, size, radius or other dimension of the fall off area may be used. Additionally or alternatively, the parameters may be changed by the user.

Generally, the size of the fall off area of a point may depend on the distance between the particular point and its neighboring points. The closer another point is to the particular point, the smaller is the area affected by a change in any of the coordinates of the particular point.

In some embodiments, the process may be applied to the two feet of a patient. Three dimensional computerized models of two feet of a patient may be received, and the user may mark the locations of the first set of points per each foot model as described above.

The locations of the points relative to each foot model, and relative to each other may be compared. For example, the difference in each dimension between any two of the points may be compared to the corresponding distance for the other foot, taking for example the direction difference. If the relations are substantially the same, for example there is no more than a predetermined deviation therebetween in any of the distances, the feet may be assumed to be symmetrical, and the orthosis model as designed for one foot may be reflected around the vertical axis, applied to the other foot as well and a corresponding orthosis may be created. The user may examine the orthosis of the other foot and enhance it as required.

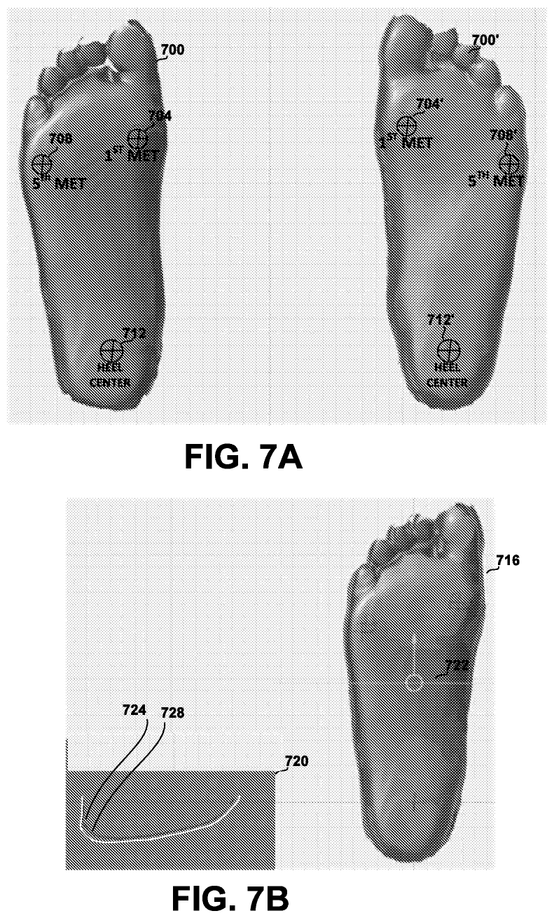

FIG. 7A shows a model 700 of the right foot of a patient, and a model 700′ of the left foot of the patient, and the indications of the first set of the anatomical points, including points 704, 708 and 712 of model 700, and points 704′, 708′ and 712′ of model 700′. The anatomical points on each foot may be used to calculate the foot rotation, using for example the heel center as a pivot point and refence point for position.

The models may then be aligned by positioning both feet in the same 3D location and rotation, and flipping one foot around its Y axis (length axis) so that both models align as if they are both scans of the same foot. In case further manual alignment is needed, the user can move and rotate each foot separately.

The system may provide an indication whether the sets of points are symmetrical, and the user may accept or override the system's indication. For example, the system may indicate that the points are symmetrical but the user may see other differences between the feet. Alternatively, although the distances may be larger than the corresponding thresholds, the user may decide that the distances are acceptable and the feet may be considered symmetrical.

If the user decided that the feet are symmetrical, whether the system so recommended or not, the user can continue working on one foot model as disclosed above, from step 116 and on. The resulting orthosis may then be produced, as well as a symmetrical one.

If the user decided that the feet are asymmetrical, whether the system so recommended or not, the user can continue working on one foot model as disclosed above, and then work on the other foot model.

In a hybrid case, the user may treat the feet as symmetrical and have symmetrical orthosis designs created, but may still introduce changes to the second orthosis model relative to the one generated upon the model.

Further features may be enabled when two feet models are available. For example, FIG. 7B shows a combined model 716 where the feet are shown on top of each other (with the symmetrical reflection) difference between the feet are highlighted. Display 720 shows together cross section 724 along line 722 of the right foot, and cross section 728 along the left foot, along the same line 722, such that the user can make better decisions about the orthosis for each foot.

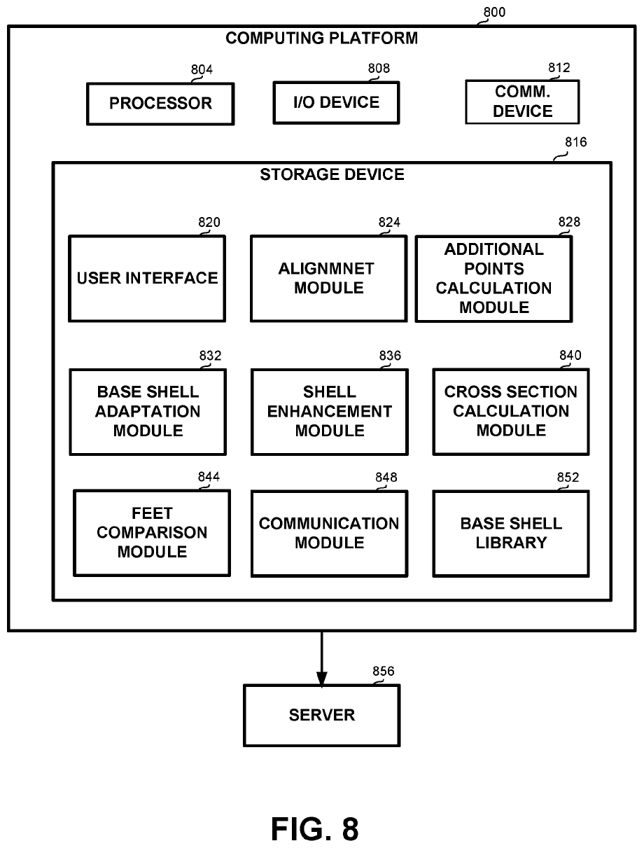

Referring now to FIG. 8 showing a block diagram of an apparatus for generating personally fitted orthoses, in accordance with some exemplary embodiments of the disclosure.

The apparatus may comprise computing platform 800. Computing platform 800 may comprise a processor 804 which may be one or more Central Processing Units (CPU), a microprocessor, an electronic circuit, an Integrated Circuit (IC) or the like. Processor 804 may be configured to provide the required functionality, for example by loading to memory and activating the modules stored on storage device 816 detailed below.

It will be appreciated that processor 804 may be implemented as one or more processors, whether located on the same platform or not. It will also be appreciated that computing platform 800 may be implemented as one or more computing platforms which may be operatively connected to each other.

Computing platform 800 may comprise input/output (I/O) device 808 such as a display, a pointing device, a touch screen, a keyboard, a speakerphone, a headset, a camera, or the like. I/O device 808 may be utilized to receive input from and provide output to a patient or another person, for example display a foot model and a shell model, receive from the user point indications, shell selection, or location updates, walk the user through the process, or the like.

Computing platform 800 may comprise a communication component 812 for communicating with other computing platforms via cellular communication, Wi-fi, Bluetooth, or the like. For example, computing platform 800 may receive foot models, shell types and default parameters, software updates, or the like, and may transmit ready shell models to be manufactured.

Computing platform 800 may comprise a storage device 816, such as a hard disk drive, a Flash disk, a Random Access Memory (RAM), a memory chip, or the like. In some exemplary embodiments, storage device 816 may retain program code operative to cause processor 804 to perform acts associated with any of the modules listed below, or steps of the methods of FIG. 1 or FIG. 2 above. The program code may comprise one or more executable units, such as functions, libraries, standalone programs or the like, adapted to execute instructions as detailed below.

Storage device 816 may comprise an application, which may be downloaded to the computing platform 800 and be used by the user for designing a shell model.

The application may comprise user interface 820, for displaying information to the user, and receiving input from the user. For example, the foot model and the shell being designed may be displayed to the user over a display device, the user may set and update locations, the user may indicate lines and the cross sections corresponding to the line may be displayed, or the like.

The application may comprise alignment module 824 for aligning the foot model according to the first set of points, as detailed in association with step 204 of FIG. 2.

The application may comprise additional points calculation module 828 for calculating the additional points based on the initial set of points, as also detailed in association with step 204 of FIG. 2.

The application may comprise base shell adaptation module 832 for adapting a base shell according to the first set of points and the additional set of points associated with the foot model. Adapting the base shell may include determining resizing and aligning parameters for the base shell (step 208), determining points on the base shell corresponding to the additional points (step 212), adjusting the z coordinate of these points according to the foot model (step 216), and adapting the fall off area of each point (step 220).

The application may comprise shell enhancement module 836 for adapting the shell after a change to one or more points of the first set of points, the additional set of points, or another parameter of the shell (steps 124, 128).

The application may comprise cross section calculation module 840 for calculating and displaying cross sections of the foot model and the shell model that correspond to a line marked by a user (step 120).

The application may comprise feet comparison module 844 for obtaining the three dimensional computerized models of two feet of a patient, and the locations of the first set of points per each foot model, and comparing them. If the points are located at substantially the same locations relative to the feet model and relative to each other, then it may be assumed that the feet are substantially symmetrical, and the process of FIG. 1 as performed for one feet may be flipped and reflected to the other feet.

Storage device 816 may comprise communication module 848 for communicating through communication component 812 with external computing platform or storage devices.

Storage device 816 may comprise or be in operative communication, through communication module 848 with base shell library 852, storing base shells for any type, shoe size, or the like, such that once the user selects a type and size, a suitable base shell is provided as a basis, and is updated based on the first set of points selected by the user. Each shell may be stored as a data structure, one or more database entries, functions, or the like, adapted to enable adapting and enhancing the design for each particular patient.

Storage device 816 may comprise or be in operative communication, through communication module with server 856, which may be collocated with computing platform 800 or remote. In some embodiments, server 856 may be a web server. Server 856 may store and transmit to one or more computing platforms 800 patient data, scans, designs, or the like, handle order status and other administrative tasks, or the like. For example, one or more shell designs may be stored, for example as a data structure or one or more database entries. Each shell design may comprise the first set of points, the additional setoff points and additional data. The shell design may be retrieved when it is required to fix the design, to design a new orthosis for the patient, or the like.

It will be appreciated that the method and apparatus may also be used for designing objects other than orthoses, such as personally adapted sandals, flip-flops, or the like, fabricated upon models of the two feet of a patient.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method for generating an orthosis shell design, comprising:
obtaining a three dimensional (3D) computerized foot model of a foot of a patient;
displaying the 3D computerized foot model over a display device;
receiving from a user locations of a first set of model points on the 3D computerized foot model, wherein the first set of model points comprising at least three points, and wherein each model point of the first set of model points is associated with a predetermined anatomical feature;
automatically adapting a base shell 3D model upon the first set of model points to obtain an adapted shell model;
automatically calculating an additional set of model points based on the first set of model points, the additional set of model points representing a shape of a foot arch of the patient; and
automatically enhancing the adapted shell model upon the first set of model points and the additional set of model points to obtain an orthosis deign for the foot.

2. The method of claim 1, further comprising:
upon receiving from the user an indication of a segment across the foot model, displaying a first cross section of the adapted shell model as enhanced and a second cross section of the 3D computerized foot model, for which the projections over a plane of the display device aligns with the segment.

3. The method of claim 1, further comprising:
receiving from the user an updated location for a point from the first set of model points or from the additional set of model points; and
updating the adapted shell model in accordance with the updated location.

4. The method of claim 1, further comprising:
receiving from the user an updated value for a pre-defined parameter for controlling a shape of the base shell 3D model or the adapted shell model; and
updating the adapted shell model in accordance with the updated value.

5. The method of claim 4, wherein the pre-defined parameter is at least one item selected from the group consisting of: scale, heel width, forefoot width, medial trim line, lateral trim line, raising of heel cup, and thickness.

6. The method of claim 1, wherein the first set of model points includes: a first metatarsal point (point A), a fifth metatarsal point (point B) and a heel center (point C).

7. The method of claim 6, wherein an X coordinate and a Y coordinate of the additional set of model points are determined based on X and Y coordinates of point A, point B and point C, and wherein a height (Z coordinate) is determined by ray casting to indicate a point on the 3D computerized foot model.

8. The method of claim 7, wherein calculating the additional set of model points includes resizing and aligning the base shell 3D model and wherein the additional set of model points describes an arch of the foot.

9. The method of claim 7, wherein the additional set of model points includes 5 points, being point 0 , point 1 , point 2, point 3 and point 4.

10. The method of claim 9, wherein the additional set of model points includes at least one point selected from the group consisting of: a midpoint between point a and a point having an X coordinate of point A and a Y coordinate of point C (point 4); a midpoint between point A and point 4 (point 2); a center point between point B and point 4 (point 3); a point having a Y coordinate of point 4 and an X coordinate of point c (point 0 ); and a midpoint between point 0 and point 4 (point 1 ).

11. The method of claim 9, wherein enhancing the adapted shell model comprises:
using point c as a reference point, determining resizing and aligning parameters for the base shell 3D model to comply with the 3D computerized foot model based on the locations of point a and point b relative to the 1^{st} and 5^{th} metatarsal points on the base shell 3D model, and adapting the base shell 3D model;
determining x and y coordinates for additional shell points on the base shell 3D model as adapted, using the resizing and aligning parameters as applied to the x and y coordinates of the additional set of model points; and
updating the base shell 3D model as adapted such that a Z coordinate of each of the additional shell points corresponds to the Z coordinate of a corresponding model point from the additional set of model points; and
enhancing the base shell 3D model as updated in a fall off area around at least one of the additional set of model points.

12. The method of claim 1, wherein the base shell 3D model is in compliance with an orthosis model selected by the user.

13. The method of claim 1, further comprising generating an orthosis based on the orthosis design.

14. The method of claim 1, wherein the 3D computerized foot model is generated by scanning a foot or a foot model or a foot impression.

15. The method of claim 1, wherein the base shell 3D model is selected from a shell library comprising basic shell designs, wherein the base shell 3D model is a data structure enabling said adapting and said enhancing.

16. The method of claim 1, further comprising:
storing the first set of model points, the additional set of model points and the adapted shell 3D model; and
retrieving the first set of model points, the additional set of model points or the adapted shell 3D model for performing a future task associated with the patient.

17. The method of claim 1, further comprising:
obtaining a second 3D computerized foot model of a second foot of the patient;
displaying the second 3D computerized foot model over a display device;
receiving from a user second locations of the first set of model points on the second 3D computerized foot model;
comparing the locations and the second locations while taking into account their symmetry to obtain a correspondence degree;
subject to the correspondence degree being acceptable, creating a second orthosis deign for the second foot, the second orthosis deign being a symmetrical reflection of the orthosis design.

18. The method of claim 17, further comprising:
subject to the correspondence degree being unacceptable, generating the second orthosis design, and upon receiving from the user an indication of a segment across the foot model, displaying a first cross section of the orthosis design and a second cross section of the second orthosis, for which the projections over a plane of the display device aligns with the segment.

19. A computerized apparatus having a processor, the processor being adapted to perform the steps of:
obtaining a three dimensional (3D) computerized foot model of a foot of a patient;
displaying the 3D computerized foot model over a display device;
receiving from a user locations of a first set of model points on the 3D computerized foot model, wherein the first set of model points comprising at least three points, and wherein each model point of the first set of model points associated with a predetermined anatomical feature;
automatically adapting a base shell 3D model upon the first set of model points to obtain an adapted shell model;
automatically calculating an additional set of model points based on the first set of model points, the additional set of model points representing a shape of a foot arch of the patient; and
automatically enhancing the adapted shell model upon the first set of model points and the additional set of model points to obtain an orthosis deign for the foot.

20. A computer program product comprising a computer readable storage medium retaining program instructions, which program instructions when read by a processor, cause the processor to perform a method comprising: obtaining a three dimensional (3D) computerized foot model of a foot of a patient;
obtaining a three dimensional (3D) computerized foot model of a foot of a patient;
displaying the 3D computerized foot model over a display device;
receiving from a user locations of a first set of model points on the 3D computerized foot model, wherein the first set of model points comprising at least three points, and wherein each model point of the first set of model points associated with a predetermined anatomical feature;
automatically adapting a base shell 3D model upon the first set of model points to obtain an adapted shell model;
automatically calculating an additional set of model points based on the first set of model points, the additional set of model points representing a shape of a foot arch of the patient; and
automatically enhancing the adapted shell model upon the first set of model points and the additional set of model points to obtain an orthosis deign for the foot.
